(19) United States
(12) Patent Application Publication
(10) Pub. No.: US 2013/0161247 A1
(43) Pub. Date: Jun. 27, 2013

(54) Apparatus for Blood Purification by Extracorporeal Circulation

(71) Applicant: INFOMED SA, Acacias (CH)

(72) Inventor: Olivier FAVRE, Thonex (CH)

(73) Assignee: INFOMED SA, Acacias (CH)

(21) Appl. No.: 13/693,232

(22) Filed: Dec. 4, 2012

(30) Foreign Application Priority Data
Dec. 21, 2011 (EP) 11010040.1

(51) Int. Cl. A61M 1/36

(52) U.S. Cl. CPC A61M 1/3621; A61M 1/3679
USPC 210/195.1; 210/194; 210/196

(57) ABSTRACT

A blood purification device includes a loop for extracorporeal circulation of blood consisting of an element (1) that extracts plasma from the blood and elements for circulating the thus extracted plasma in elements for purifying the plasma (3, 4, 5, 6 or 8). The device includes in addition a column (2) in the blood circulation loop.

### FIELD OF THE INVENTION

This invention relates to a device for purification of the blood of patients who exhibit a pathology that makes it necessary to reduce the circulating amounts of several substances having different physico-chemical characteristics. Crohn's disease, rheumatoid arthritis, certain forms of psoriasis or even Behcet's disease belong, i.a., to this category because the immune and inflammatory systems interact to create chronic inflammations that progress until greatly reducing the quality of life of patients.

### BACKGROUND OF THE INVENTION

The interaction mechanisms between the leukocytes and the antibodies, on one hand, for the immune system and the cytokines, on the other hand, for the inflammatory system have been studied and are the object of several publications that clarify certain aspects of these interactions while demonstrating their incredible complexity. The sum of knowledge of all of the studies in question ultimately describes only the existing mechanisms in a simplified manner. The importance of each substance in the inflammation as well as their mutual stimulation to multiply pathologically has been demonstrated, however; thus, among the different existing leukocytes, these are primarily the monocytes and the granulocytes that are involved in the pathologies mentioned above, with the lymphocytes playing a minor role.

In the most serious cases of pathologies described above, a purification of the blood of patients by extracorporeal circulation is implemented. In the conventional treatment modes, two approaches exist whose purpose is primarily to reduce the immune reaction by purifying leukocytes or antibodies. The purification of cytokines has not been practiced in these cases.

To remove leukocytes from blood, there are two techniques, one proceeding by filtration and the other by adsorption. These methods were developed in the 1990s. In the two cases, the purification element is placed on the circuit for extracorporeal circulation of blood, which runs through said element and leaves it with a reduced concentration of leukocytes relative to the initial value in the element. This element that has only one entrance and one exit for the blood and retains certain specific substances is called “column.” In the case of a column that acts by filtration, the fact is used that the leukocytes are the largest substances contained in the blood to retain them mechanically, whereas the red globules and other proteins run through the column. One example of such an element is the Cellsorba of the Asahi Kasei Company that was technically described in the patent U.S. Pat. No. 4,476,023 but for a different application from the one presented here. In the case of the element acting by adsorption, the affinity of leukocytes for the material contained in the element is used, material to which the leukocytes “adhere” whereas the other elements remain free to circulate in the blood. An example of such an element is the Adacolumn of the Jimro Company, object of patent U.S. Pat. No. 6,498,007.

These two purification methods exhibit analogous clinical results despite leukocyte retention rates that are different. It should be noted that centrifuging techniques are also used occasionally but with inferior clinical results.

To withdraw antibodies from blood, three techniques exist. Firstly, the plasmatic exchange, in which the plasma that is extracted from the blood is thrown out while a compensation fluid that contains albumin is injected to compensate for losses; secondly, the cascade filtration during which the plasma that is extracted from the blood runs through a filter that retains the antibodies before being returned to the blood; and thirdly, the plasma adsorption that uses an adsorbent column that is designed to retain the antibodies on the plasma circuit.

These three techniques were invented in the 1970s. The plasmatic exchange is practiced today at a rate of several hundreds of thousands of treatments per year in the world. Several devices exist for carrying out the plasmatic exchange: they separate the plasma from the blood by filtration or by centrifuging, whereas the balancing of the volumes between the thrown-out plasma and the compensation solution is done by using either balances or the turn number of the respective pumps. The cascade filtration is used today at a rate of several tens of thousands of treatments per year. Several devices exist that make it possible to implement this technique. The plasma adsorption is practiced at a rate of several thousands of treatments per year with the different available adsorbent column models. Again, these three methods have analogous clinical results that are overall slightly inferior to those consisting in withdrawing leukocytes.

The methods for blood purification by extracorporeal circulation that are described above are still ineffective for the most seriously ill patients as well as for 20 to 50% of moderately ill patients. To remedy this drawback and to be able to treat all of the patients with a more elaborate device for blood purification by extracorporeal circulation, the object of this invention is to be carried out. The reason for the limits of existing devices is that they make it possible to withdraw only one of the elements at a time, either the leukocytes, the antibodies, or the cytokines; the result is that the other elements, which remain in surplus, next activate the regeneration of what has been withdrawn. It would therefore be useful to be able to withdraw several categories of elements in a single blood passage so as to prevent this cascade of reactions that prevents the pathology from being cured and even reduced.

The fact that in practice, the available systems withdraw only a single category of substance comes both from an accumulation of differences in their physico-chemical characteristics and therefore in the possible means for withdrawing them from the blood. The leukocytes, owing to their large size, are not present in the plasma and should therefore be withdrawn directly from the blood. The antibodies can, in theory, be adsorbed directly in the blood, but it is preferred to withdraw them from plasma to avoid interactions between adsorbent material and blood. They are thus withdrawn from the blood either by throwing out the plasma (plasmatic exchange) or by cascade filtration or by adsorption. The cytokines are preferably withdrawn from plasma by adsorption because the volume to be treated in order to be effective is much larger than that which the plasmatic exchange allows. Finally, even if, a priori, all of these substances can be withdrawn by adsorption, the usable materials and their production methods are different.

### OBJECTS AND SUMMARY OF THE INVENTION

The object of this invention is to eliminate, at least partly, the above-mentioned drawbacks.

To do this, the object of this invention is a device that makes it possible to withdraw from patients' blood no longer only a category of substance involved in the cascade of reactions leading to pathology, as is the case in the techniques used to date, but simultaneously at least two different categories such as, for example, the leukocytes and the antibodies or the leukocytes and the cytokines or the antibodies and the cytokines and even the leukocytes, the antibodies, and the cytokines.

There are certain devices that withdraw different substances owing to their nature but that are involved in the same pathology. It is possible to cite the patent EP2295092 of Bellco that comprises a hemofilter and a hemodialyzer on the loop for extracorporeal circulation of blood, with the first, the hemofilter, making it possible to withdraw water and toxins of a high molecular weight, while the second, the hemodialyzer, does the same with the small molecules. Another example is the patent U.S. Pat. No. 4,683,889 that describes a device that comprises—on the blood loop—a UV irradiation device followed by a centrifuging device that is used to withdraw lymphocytes, the unit being designed to combat leukemia. Another example is the patent application U.S. Pat. No. 6,193,681 that comprises—on the blood loop—a UV irradiation device followed by a hemofilter for the treatment of sepsis. The patent US20040143207 describes a blood loop that comprises a hemofilter and an oxygenator, with this invention making it possible to treat patients that are ill with renal and respiratory failure.

It thus is seen that various devices already make it possible, by the use of several means of actions located in series on the loop for extracorporeal circulation of blood, to withdraw substances that have different physico-chemical characteristics.

### BRIEF DESCRIPTION OF THE DRAWINGS

FIGS. 1-3 depicted different embodiments of the blood circulation loops according to the Invention.

FIG. 4 shows a device in which the plasma is thrown out into a tank (8) whereas a compensation fluid contained in a tank (7) is added into the blood circulation circuit.

FIG. 5 shows a system in which the plasma runs through a filter (3) for withdrawing antibodies therefrom.

FIG. 6 shows a device that comprises a filter (3) that is designed to retain antibodies followed by a cytokine adsorption element (4).

FIG. 7 exhibits a version of the device in which two adsorption columns (4, 5) are placed on the plasma line, one retaining antibodies and the other cytokines.

FIG. 8 comprises a single cartridge (6) that is designed in such a way as to retain both antibodies and cytokines.

### DETAILED DESCRIPTION OF THE DRAWINGS

A device according to this invention has, as shown in FIGS. 1 to 8, a loop for extracorporeal circulation of blood on which there are placed an element that makes it possible to extract plasma from the blood and a column that is used, for example, to retain leukocytes. A plasma circulation circuit that comprises means that make it possible to eliminate other substances, for example antibodies, cytokines, or both, completes the device.

An essential advantage of this invention resides in the fact that it makes it possible, relative to the existing techniques, to significantly reduce the quantity and the diversity of substances that intervene in the pathologies described above, thus allowing a greater clinical effectiveness.

The variant embodiments according to the invention are shown in FIGS. 1 to 8. They all have a loop for extracorporeal circulation of blood that consists of a conduit for extracting blood from patient (a) on which is found a blood-plasma separation element (1) connected by a conduit (c) to a column (2), with the blood then being returned to the patient via conduit (b). A circuit of this type is, of course, completed by pressure measuring means, one or more pumps, an air detector, all elements that are described in numerous publications and technical standards that are applied to this in all known variants without affecting its primary characteristic.

Different configurations of the blood circulation loop are possible and all are suitable for producing a device according to this invention. By way of example, it is possible to cite a device that comprises: a blood-extracting needle, a tube equipped with a connector for reading the extraction pressure connecting said needle to a peristaltic pump that forces blood through the blood loop, a filter with hollow fibers having a porosity such that the plasma can be extracted from the blood, a blood return tube connected to a blood return needle and comprising a bubble trap and a connector for reading the return pressure. The return tube runs into an air bubble detector that follows a clamp, with the unit making it possible to prevent a possible injection of air into the patient. Variants of such a device can comprise a double-lumen catheter by replacing needles and/or a blood/plasma separator that acts by centrifuging in replacement of the filter. Such devices have existed since the 1970s-1980s. In one or the other version of this known circulation loop, a blood circulation loop according to the invention will not comprise one but rather two purification elements, a plasma filter 1 and a column 2 retaining specific substances, such as, for, example, leukocytes.

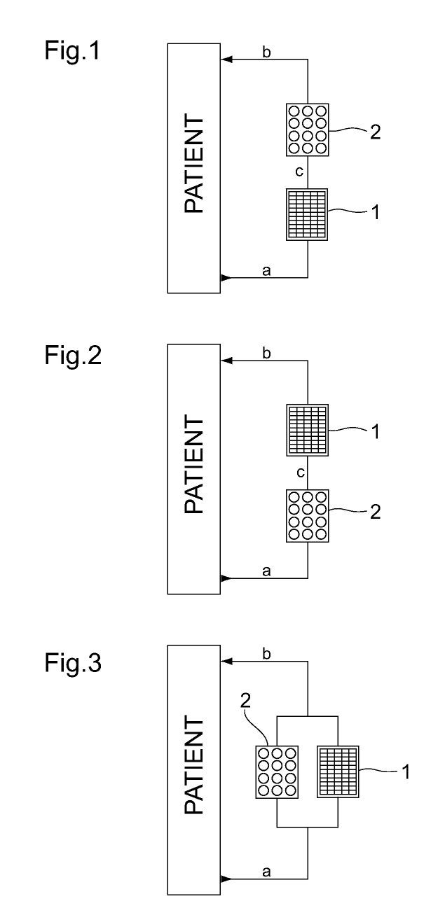

The elements 1 and 2 can be placed arbitrarily on the blood loop without this influencing the operation of this invention. The element 2 can, for example, be positioned downstream from the element 1 (FIG. 1), upstream from element 1 (FIG. 2), or the two elements can be placed in parallel (FIG. 3), variants that are all part of this invention.

One or more means for withdrawing substances from the extracted plasma are added to this blood circulation loop that comprises a column 2 that retains specific elements and an element 1 that extracts plasma.

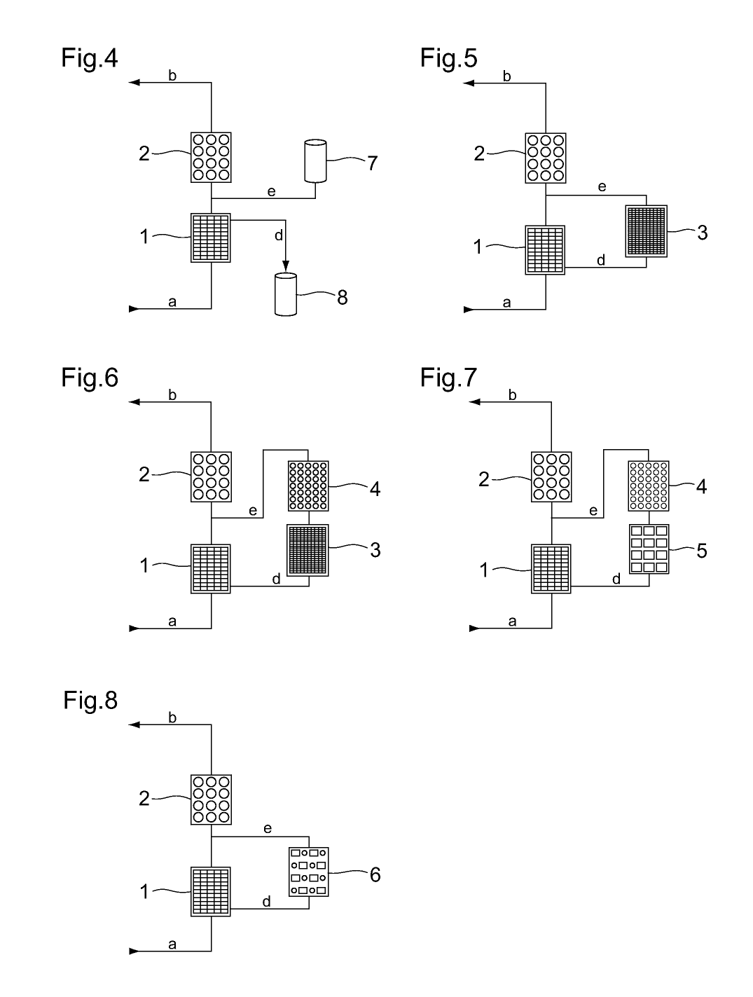

The plasma circulation loop of a device that operates in accordance with this invention, shown in one of FIGS. 4 to 8, consists of a tube (d) that makes it possible to circulate the plasma from the blood-plasma separator 1, known circulation means that are not shown of the plasma in the plasma loop, one or more elements (3, 4, 5, 6, 8) designed to withdraw various substances from the plasma and a return tube (e) of the plasma that is thus purified in the blood circulation loop of the patient.

FIG. 4 shows a device in which the plasma is thrown out into a tank (8) whereas a compensation fluid contained in a tank (7) is added into the blood circulation circuit.

FIG. 5 shows a system in which the plasma runs through a filter (3) for withdrawing antibodies therefrom.

FIG. 6 shows a device that comprises a filter (3) that is designed to retain antibodies followed by a cytokine adsorption element (4).

FIG. 7 exhibits a version of the device in which two adsorption columns (4, 5) are placed on the plasma line, one retaining antibodies and the other cytokines.

FIG. 8 comprises a single cartridge (6) that is designed in such a way as to retain both antibodies and cytokines.

These plasma loops that are illustrated in FIGS. 4 to 8 in a configuration of the extracorporeal circulation loop according to FIG. 1, where the extraction and the return of plasma are done upstream from column 2, can, of course, also be applied to the loops for extracorporeal circulation of blood according to FIGS. 2 and 3. Likewise, the return of plasma into the blood circulation loop can be done on any segment (a), (b), or (c).

1. Blood purification device that comprises a loop for extracorporeal circulation of blood that consists of an element that extracts plasma from the blood and means for circulation of the thus extracted plasma in plasma purification means, characterized in that it comprises in addition one column in said blood circulation loop.

2. Device according to claim 1, wherein the column retains leukocytes.

3. Device according to claim 1, wherein the column and the element are placed in series on the blood circulation loop.

4. Device according to claim 1, wherein the column and the element are placed in parallel on the blood circulation loop.

5. Device according to claim 1, wherein the plasma is thrown out into a tank and then fully or partially compensated by a substitution liquid contained in a tank.

6. Device according to claim 1, wherein the extracted plasma is circulated through a filter and then returned into the blood circulation loop.

7. Device according to claim 1, wherein the extracted plasma is circulated through a filter and an adsorbent cartridge and then returned into the blood circulation loop.

8. Device according to claim 1, wherein the extracted plasma is circulated through two adsorbent cartridges and then returned into the blood circulation loop.

9. Device according to claim 1, wherein the extracted plasma is circulated through a single adsorbent cartridge retaining antibodies and cytokines and then returned into the blood circulation loop.
